(19) United States
(12) Patent Application Publication
(10) Pub. No.: US 2023/0181048 A1
(43) Pub. Date: Jun. 15, 2023

(54) PHYSICAL CONDITION CHANGE DETECTION DEVICE, PHYSICAL CONDITION CHANGE MANAGEMENT PROGRAM, AND PHYSICAL CONDITION CHANGE MANAGEMENT SYSTEM

(71) Applicants: Takunori Shimasazki, Hyogo (JP); METS INC., Tokyo (JP); EMUZU LTD., Hyogo (JP)

(72) Inventors: Takunori Shimazaki, Hyogo (JP); Mitsuhiro Fukuda, Tokyo (JP); Masakazu Aoki, Hyogo (JP)

(73) Assignees: Takunori Shimazaki, Hyogo (JP); METS INC., Tokyo (JP); EMUZU LTD., Hyogo (JP)

(21) Appl. No.: 17/925,408

(22) PCT Filed: May 18, 2021
(86) PCT No.: PCT/JP2021/018820

§ 371 Date: Nov. 15, 2022

(30) Foreign Application Priority Data
May 19, 2020 (JP) 2020-087745

(51) Int. Cl. A61B 5/0205; A61B 5/00

(52) U.S. Cl. CPC A61B 5/02055; A61B 5/747

(57) ABSTRACT

A physical condition change detection device for detecting a change in physical condition of a subject includes a body-surface-side biological information detection unit that is configured to detect a variation in biological information obtained from a body surface of the subject and that is to face the body surface of the subject and an outside thermal variation detection unit that faces away from the body-surface-side biological information detection unit and that is configured to detect a thermal variation in an outside area separated from the body surface of the subject. The physical condition change detection device further includes a transmit unit configured to provide control to transmit detection data of the body-surface-side biological information detection unit and the outside thermal variation detection unit to the outside at every given time.

### TECHNICAL FIELD

The present invention relates to a technology for detecting changes in physical condition.

### BACKGROUND ART

At work sites such as construction sites and building sites, workers sometimes work in high temperature environments, and thus, it is necessary to provide measures for workers to prevent heatstroke. A known indicator for determining the risk of heatstroke is the Wet Bulb Globe Temperature (WBGT), which is calculated based on wet-bulb temperature, globe temperature, and dry-bulb temperature. WBGT is used as a reference value of the risk of heatstroke, calculated with consideration of effects of humidity and radiant heat as well as air temperature.

As examples of related technologies of determining the risk of heatstroke based on WBGT, PTL 1 discloses a system configured to calculate the value of WBGT based on results of measuring the room temperature and humidity in a room in which a subject is present, determine whether the risk of heatstroke exists based on the calculated result, and accordingly output a heatstroke alert. PTL 2 discloses a system configured to determine the risk of heatstroke of a subject based on values measured with WBGT sensors for measuring air temperature and humidity; to determine the risk of heatstroke properly for individual target people, the WBGT sensors are provided at both inner and outer parts of the things worn by the subject, such as clothes and a helmet.

### CITATION LIST

### Patent Literature

PTL 1: Japanese Unexamined Patent Application Publication No. 2017-168098
PTL 2: Japanese Unexamined Patent Application Publication No. 2020-016528

### SUMMARY OF INVENTION

### Technical Problem

The risk that heatstroke may occur can vary depending on, for example, the body size, physical constitution, and physical condition of the subject and the environment and condition in which the subject is present. To manage physical condition of the subject, it is preferable to detect changes in physical condition such as hypothermia and physical exhaustion, as well as heatstroke. Overall, to determine changes in physical condition of a subject, it is desirable to detect changes in physical condition based on variable factors of changes in physical condition.

The present invention has been made in consideration of the above circumstances, and an object thereof is to detect changes in physical condition of a subject.

### Solution to Problem

According to an aspect of the present invention, a physical condition change detection device for detecting a change in physical condition of a subject includes a body-surface-side biological information detection unit configured to detect a variation in biological information obtained from a body surface of the subject and an outside thermal variation detection unit configured to detect a thermal variation in an outside area separated from the body surface of the subject.

According to an aspect of the present invention, it is possible to detect a change in physical condition of a subject based on a variation in biological information obtained from the body surface of the subject and a thermal variation in an outside area separated from the body surface of the subject.

According to an aspect of the present invention, a physical condition change management program causes at least one processor to function as at least a receive unit and a determination unit. The receive unit is configured to obtain measurement data indicating a variation in biological information obtained from a body surface of a subject and a thermal variation in an outside area separated from the body surface. The determination unit is configured to determine whether there is a risk of occurrence of the change in physical condition of the subject based on at least the measurement data. With this configuration, it is possible to manage changes in physical condition of the subject.

According to an aspect of the present invention, a physical condition change management system includes at least one processor and a physical condition change management program causing the processor to function as at least a receive unit and a determination unit. The receive unit is configured to obtain measurement data indicating a variation in biological information obtained from a body surface of a subject and a thermal variation in an outside area separated from the body surface. The determination unit is configured to determine whether there is a risk of occurrence of the change in physical condition of the subject based on at least the measurement data. With this configuration, it is possible to manage changes in physical condition of the subject.

### Advantageous Effects of Invention

According to an aspect of the present invention, it is possible to detect changes in physical condition of the subject.

### BRIEF DESCRIPTION OF DRAWINGS

FIGS. 1(A) and 1(B) are perspective views illustrating a basic structure of a physical condition change detection device according to an embodiment of the present invention.

FIGS. 2(A) and 2(B) are perspective views illustrating examples of an operating unit of the physical condition change detection device according to the embodiment of the present invention.

FIG. 3 illustrates a subject wearing the physical condition change detection device according to the embodiment of the present invention.

FIG. 4 is a block diagram illustrating a basic configuration of functions of the physical condition change detection device according to the embodiment of the present invention.

FIG. 5(A) illustrates an arrangement of main parts of the physical condition change detection device according to the embodiment of the present invention. FIG. 5(B) illustrates an operation of the physical condition change detection device according to the embodiment of the present invention.

FIGS. 6(A) to 6(C) illustrate arrangements of main parts of the physical condition change detection device according to other embodiments of the present invention.

FIG. 7 is a block diagram illustrating a basic configuration of a physical condition change management system according to an embodiment of the present invention.

FIG. 8 is a block diagram illustrating a specific configuration of main parts of the physical condition change management system according to the embodiment of the present invention.

FIGS. 9(A) and 9(B) present an example of questionnaires that are transmitted to a subject using the physical condition change management system according to the embodiment of the present invention.

FIG. 10 illustrates pressure changes by five levels of RGB colors of light of a pulse wave sensor fixed to a fingertip.

FIG. 11 illustrates differences of pulse waveform and motion artifact among different wavelengths.

### DESCRIPTION OF EMBODIMENTS

Hereinafter, a preferred embodiment of the present invention will be described in detail. It is noted that the embodiments described below should not unduly limit the scope of the present invention described in the claims, and all the configurations described in the embodiments are not necessarily essential to the present invention as means for solving the problems.

Basic Structure of Physical Condition Change Detection Device 100

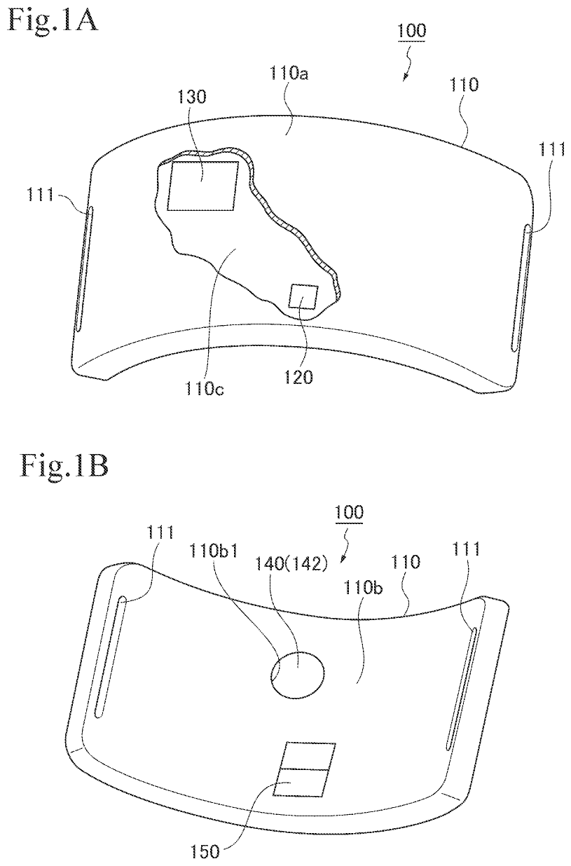

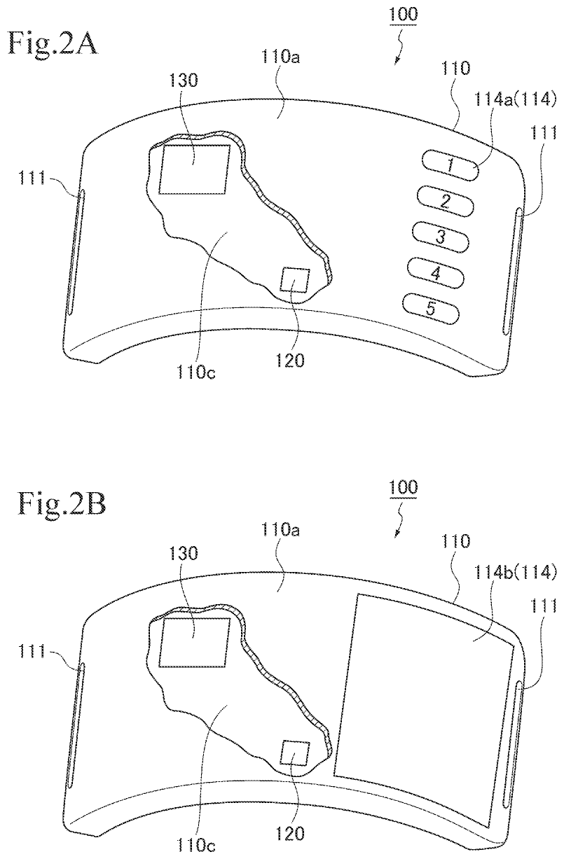

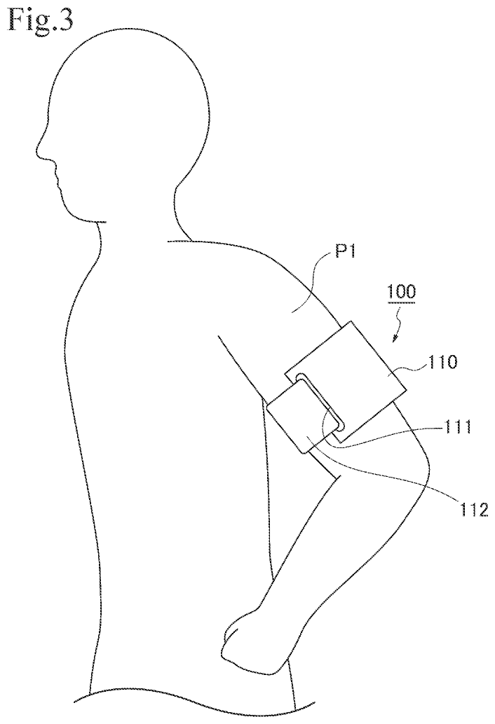

Firstly, a basic structure of a physical condition change detection device according to an embodiment of the present invention will be described with reference to the drawings. FIG. 1(A) is a perspective view of the physical condition change detection device when viewed from outside. FIG. 1(B) is a perspective view of the physical condition change detection device when viewed from inside. FIGS. 2(A) and 2(B) are perspective views of the physical condition change detection device, illustrating examples of an operating unit of the physical condition change detection device. FIGS. 1(A), 2(A), and 2(B) illustrate the physical condition change detection device in the state in which a front surface of a casing is partially cut away so that the arrangement of components in the casing is viewable.

The physical condition change detection device 100 of the present embodiment is usable to monitor a person in an environment in which their physical condition can change, who is regarded as a “subject”, and detect changes in physical condition such as heatstroke, hypothermia, physical exhaustion, and overexertion. Examples of the subject include (1) a “worker who works outside”, for example an on-site worker at a construction site or building site or a police officer directing traffic, (2) a “worker who works inside”, for example a worker who works in a hospital or business firm, (3) a “person who stays outside”, for example a preschooler, student, or adult who does something in an outside space such as a playground, and (4) a “person who stays inside”, for example an elderly person who is bedridden or requires nursing care.

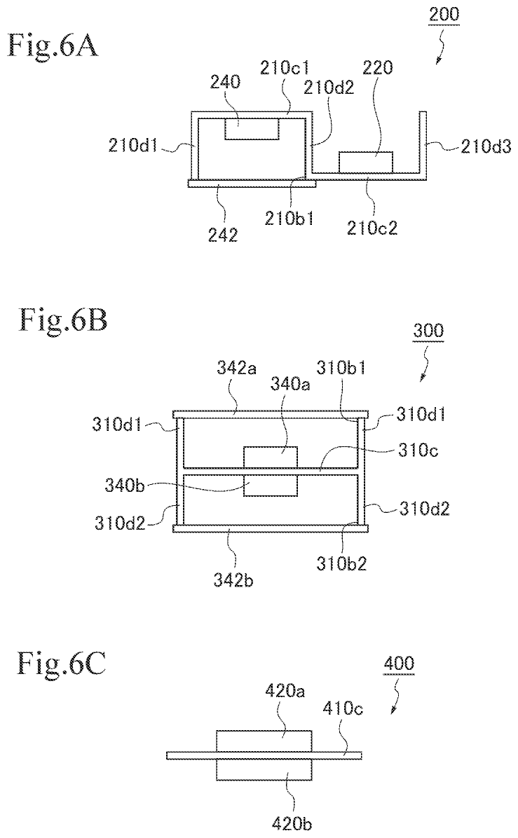

The physical condition change detection device 100 includes a temperature and humidity sensor 120, an acceleration sensor 130, a thermopile sensor 140, and a pulse wave sensor 150, which are housed in a casing 110. The arrangement of these sensors will be described later with reference to FIG. 6.

The casing 110 is a resin object. The resin used may be a hard resin or soft resin; otherwise, a hard resin and a soft resin may be used together to individually form different parts. The casing 110 forms a curve. As a result, the casing 110 fits an upper arm of a subject when the subject wears the casing 110 (FIG. 3). A battery (not illustrated in the drawings), a communication unit 113, an operating unit 114, a display unit 115, a read-only memory (ROM) 116, a random-access memory (RAM) 117, and a control unit 160 (refer to FIG. 3) are provided in the casing 110. The operating unit 114 and the display unit 115 are exposed to the outside of the casing 110.

The temperature and humidity sensor 120 is provided at a substrate 110c in the casing 110, as illustrated in FIG. 1(A). The temperature and humidity sensor 120 is provided on a front surface side of the substrate 110c. The temperature and humidity sensor 120 operates as an “outside thermal variation detection unit” for detecting thermal variation in an outside area with respect to the substrate 110c, in other words, an outside area separated from the body surface of the subject wearing the physical condition change detection device 100. The temperature and humidity sensor 120 has a function of measuring at least the temperature and humidity in an outside area with respect to the substrate 110c to detect thermal variation in an outside area with respect to the substrate 110c. This means that the temperature and humidity sensor 120 has a function of measuring the temperature and humidity in an area on a front surface 110a side with respect to the casing 110 as the temperature and humidity in an outside area separated from the body surface of the subject. The temperature and humidity sensor 120 is capable of measuring at least the temperature and humidity in an area inside a garment worn by the subject.

The acceleration sensor 130 has a function of detecting the momentum of the subject wearing the physical condition change detection device 100. Specifically, the acceleration sensor 130, for example, outputs signals corresponding to acceleration rates in three kinds of axial directions (top-to-bottom, left-to-right, and depth directions) to detect the momentum of the subject. The acceleration sensor 130 is provided on the front surface side of the substrate 110c, but it is sufficient that the acceleration sensor 130 be provided in the casing 110. The acceleration sensor 130 may thus be provided on the back surface side of the substrate 110c.

The thermopile sensor 140 operates as a “body-surface-side biological information detection unit” for detecting variations in biological information obtained at the body surface of the subject. The thermopile sensor 140 is a non-contact temperature sensor capable of detecting an absolute value of temperature using the effect of thermoelectromotive force; the thermopile sensor 140 detects variations in radiant heat, which is one kind of variations in biological information obtained at the body surface of the subject. As such, the thermopile sensor 140 has a function of a “radiant heat detection unit”. The thermopile sensor 140 is provided on the back surface side of the substrate 110c apart from the body surface of the subject, and is to face the body surface of the subject across a space. In other words, the thermopile sensor 140 is positioned such that the detection direction points toward the body surface. The temperature and humidity sensor 120 described above is positioned such that the detection direction is opposite to the detection direction of the thermopile sensor 140; in other words, the temperature and humidity sensor 120 faces the outside when viewed from the body surface.

A cavity 110b1 is provided at the back surface 110b of the casing 110; the cavity 110b1 is shaped as a tubular wall extended inside the casing 110. The thermopile sensor 140 is positioned inside the cavity 110b1. An end of the cavity 110b1 on the substrate 110c side is in contact with the substrate 110c. The substrate 110c is fixed to the casing 110 with a bolt, which is not illustrated in the drawings. A thermally conductive sheet 142 having thermal conductivity is disposed on the cavity 110b1 to cover the opening of the cavity 110b1. The thermally conductive sheet 142 is provided to block entry of sweat or liquid from the body surface and to increase conductivity of heat from the body surface of the subject to the thermopile sensor 140. Details of the thermally conductive sheet 142 will be described later.

The pulse wave sensor 150 constitutes the “body-surface-side biological information detection unit” for detecting variations in biological information obtained at the body surface of the subject. The pulse wave sensor 150 has a function of measuring the heart rate of the subject. In the present embodiment, the pulse wave sensor 150 is provided on the back surface 110b side of the casing 110 as illustrated in FIG. 1(B). As such, the pulse wave sensor 150 can measure the heart rate of the subject by photoplethysmography. To measure the heart rate, the pulse wave sensor 150 is configured such that light is emitted by a color light-emitting diode (LED) of RGB colors, which are the three primary colors of light, and the emitted light is received by a color sensor formed by an RGB photodiode and other elements.

Specifically, the pulse wave sensor 150 is implemented by a reflection-type pulse wave sensor including a color LED serving as a light-emitting source, provided on the back surface side of the substrate 110c, and a color sensor serving as a light receiving unit; the color LED and the color sensor are arranged in parallel with each other. The pulse wave sensor 150 of the present embodiment is capable of measuring the heart rate by illuminating a living body with light emitted by the color LED, receiving the reflected light by the color sensor, and sensing changes with time of the blood flow rate (changes in volume of a blood vessel), which are caused along with pulsations of the heart, to measure pulse wave signals. The pulse wave sensor 150 is not limited to a reflection-type pulse wave sensor and may be a transmission-type pulse wave sensor.

Because the color LED in the pulse wave sensor 150 is of RGB colors, which are the three primary colors of light, it is possible to obtain various colors, for example from yellow to light blue. Because the color sensor in the pulse wave sensor 150 is formed by an RGB photodiode and other elements, it is possible to distinguish many colors as human vision can. This means the pulse wave sensor 150 is configured such that the wavelength of light emitted by the color LED is changeable, and the color sensor is capable of receiving emitted light of different wavelengths.

The pulse wave sensor 150 is configured such that the color LED can alter the color of light by changing the wavelength of light to emit, for example, red, orange, yellow, green, blue, or purple light, and the color sensor serving as the light receiving unit can receive reflected light of different wavelengths. As a result, while the subject moves, the color LED emits light of a color suitable for conditions such as individual differences and the pressure for fixing the pulse wave sensor 150, and the color sensor receive the emitted light. In this manner, the heart rate can be detected by photoplethysmography with little or no effect of the conditions.

A plurality of pulse wave sensors 150 may be provided. For example, two pulse wave sensors 150 may be provided. Specifically, a first pulse wave sensor 150 is provided on the back surface 110b side of the casing 110. A second pulse wave sensor 150 is provided in the casing 110, spaced apart by a given distance from the first pulse wave sensor 150. Of the casing 110, the first pulse wave sensor 150 is provided at a surface that is to be in contact with the body surface of the subject. As such, the first pulse wave sensor 150 can be brought into close contact with the skin of the subject wearing the physical condition change detection device 100 and thus usable as a general heart rate meter (Photo Plethysmo Gram (PPG)). By contrast, the second pulse wave sensor 150 is provided in the casing 110 apart from the surface of the casing 110 having the first pulse wave sensor 150. As such, the second pulse wave sensor 150 can be placed without contact with the skin of the subject and thus usable as a motion artifact (MA) sensor.

As described above, because the two pulse wave sensors 150 are provided close to each other, while the subject is at rest, no motion artifact occurs, and the first pulse wave sensor 150 operating as a PPG outputs only the waveform of a pulse wave. By contrast, while the subject moves, the first pulse wave sensor 150 operating as a PPG outputs a waveform formed by mixing motion artifacts with a pulse wave, and the second pulse wave sensor 150 operating as a MA sensor outputs only the motion artifacts. These outputs are processed with an adaptive filter, which is not illustrated in the drawings, to extract only the difference, so that only the pulse wave can be extracted. With this configuration, it is possible to accurately detect the heart rate while the subject moves.

The operating unit 114 is provided on the front surface side of the casing 110. The operating unit 114 has a function of inputting predetermined instructions and various kinds of data to operate the physical condition change detection device 100. The operating unit 114 may be implemented by, for example, input buttons 114a designed as push buttons as illustrated in FIG. 2(A) or a touch panel 114b as illustrated in FIG. 2(B). Both the input buttons 114a and the touch panel 114b may be provided together. With the operating unit 114, at least “subjective data” of the subject can be inputted. Details of “subjective data” and an operation of inputting the subjective data with the operating unit 114 will be described later.

Slits 111 for connecting a belt 112 is provided on both sides (ends in the longitudinal direction) of the casing 110. As illustrated in FIG. 3, in the state in which the belt 112 is passed through the slits 111 of the casing 110, the casing 110 can be attached to an upper arm of a subject P1. The physical condition change detection device 100 may be attached to any part other than upper arms of the subject P1.

Functional Configuration of Physical Condition Change Detection Device 100

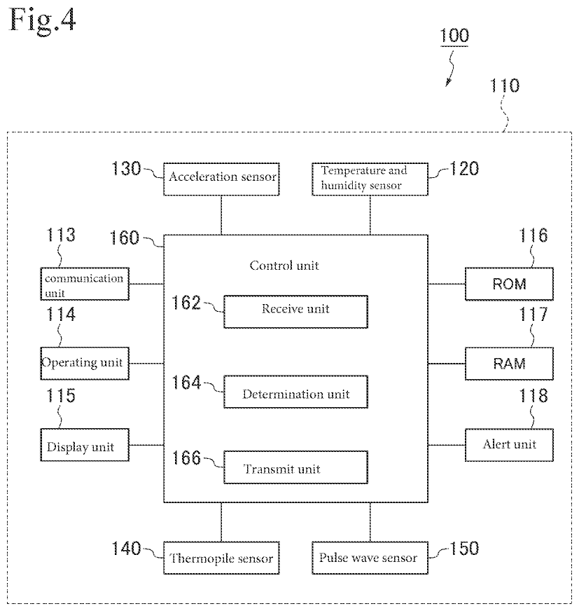

The following describes a functional configuration of the physical condition change detection device 100. FIG. 4 is a block diagram illustrating a basic configuration of functions based on hardware components of the physical condition change detection device 100 of the present embodiment.

The physical condition change detection device 100 includes a plurality of sensors of the temperature and humidity sensor 120, the acceleration sensor 130, the thermopile sensor 140, and the pulse wave sensor 150 described above. The physical condition change detection device 100 also includes, as illustrated in FIG. 4, the communication unit 113, the operating unit 114, the display unit 115, the control unit 160, the ROM 116, the RAM 117, and an alert unit 118.

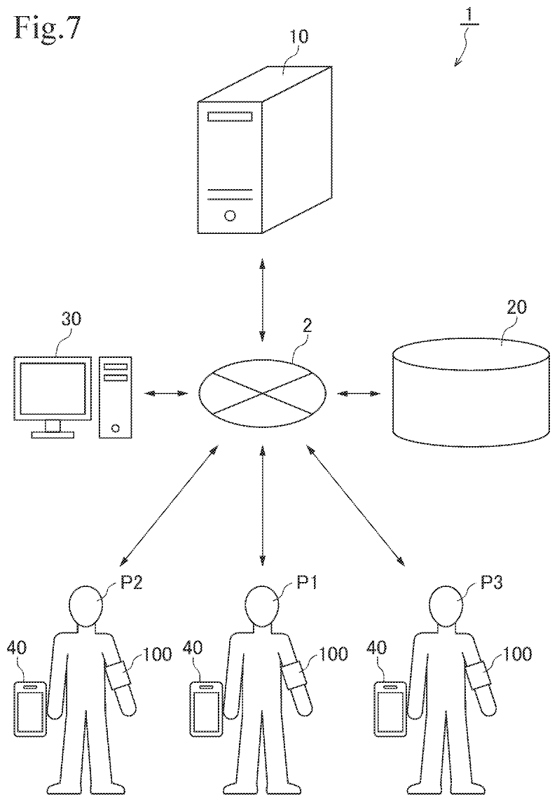

The communication unit 113 has a function as an interface used when data is exchanged from the outside through a network 2 (refer to FIG. 7). The communication unit 113 can provide communication according to a wireless communication standard, such as long term evolution (LTE), Wi-Fi (registered trademark), or Bluetooth (registered trademark). The operating unit 114 is implemented by, for example, the input buttons 114a or the touch panel 114b (refer to FIG. 2) and configured to input predetermined instructions and data to the control unit 160. In the present embodiment, the operating unit 114 has a function of inputting of subjective data created by collecting, for example, answer data to questionnaires about changes in physical condition of a subject and personally identifiable information including sex, age, and body mass index (BMI). The display unit 115 has a function of outputting, for example, results of operations performed by the control unit 160 and inputs entered using the operating unit 114 by displaying. The display unit 115 may be implemented by, for example, a liquid crystal screen. The alert unit 118 has a function of notifying the subject of information by using sound or vibration. Details of an operation of providing an alert about changes in physical condition by the alert unit 118 will be described later.

The control unit 160 has a function of controlling operations of the elements included in the physical condition change detection device 100 by running various programs stored in the ROM 116. The control unit 160 also has a function of, when providing control to execute these various operations, causing the RAM 117, which temporarily stores data, to store required data and other data as appropriate. As a result, control by the control unit 160 enables operations including an operation of accessing the ROM 116 and the RAM 117, an operation of displaying data with the display unit 115, an operation on the operating unit 114, and an operation of exchanging various kinds of information using the communication unit 113 as an interface through the network 2 when communicating with the outside.

The control unit 160 may also have a function of receiving detection data from sensors of the temperature and humidity sensor 120, the acceleration sensor 130, the thermopile sensor 140, and the pulse wave sensor 150, determining, for example, whether a change in physical condition of the subject has occurred based on the detection data, and transmitting the determination result to the outside. The control unit 160 includes, as illustrated in FIG. 4, a receive unit 162, a determination unit 164, and a transmit unit 166.

The receive unit 162 has a function of controlling reception of detection data from the individual detection sensors of the temperature and humidity sensor 120, the acceleration sensor 130, the thermopile sensor 140, and the pulse wave sensor 150. The receive unit 162 also has a function of controlling reception of various kinds of data from external devices. The receive unit 162 also has a function of controlling reception of questionnaire data about changes in physical condition of the subject, transmitted from external devices to the physical condition change detection device 100.

The determination unit 164 has a function of providing determination required when various operations of the physical condition change detection device 100 are performed. Specifically, the determination unit 164 has a function of determining whether various kinds of data are exchanged via the communication unit 113 of the physical condition change detection device 100 with external devices. The determination unit 164 may also have a function of determining whether a change in physical condition of the subject has occurred, by computationally processing detection data obtained by the individual detection sensors of the temperature and humidity sensor 120, the acceleration sensor 130, the thermopile sensor 140, and the pulse wave sensor 150.

Specifically, with reference to the temperature data and humidity data about the outside of the casing 110, received from the temperature and humidity sensor 120, and the detection data of radiant heat from the body surface of the subject, received from the thermopile sensor 140, resultant differences are calculated; based on the resultant differences, the determination unit 164 determines whether the subject is at a risk of developing heatstroke. At this time, the risk is determined in a comprehensive manner with consideration of the momentum of the subject detected by the acceleration sensor 130 and the heart rate of the subject detected by the pulse wave sensor 150. In this manner, it is possible to more accurately detect changes in physical condition based on variable factors of changes in physical condition that may vary depending on, for example, the body size, physical constitution, and physical condition of the subject and the environment and condition in which the subject is present.

The transmit unit 166 has a function of controlling transmission of various kinds of data to external devices. In the present embodiment, the transmit unit 166 provides control to transmit detection data from the individual detection sensors of the temperature and humidity sensor 120, the acceleration sensor 130, the thermopile sensor 140, and the pulse wave sensor 150 to, for example, an administrator terminal 30 (refer to FIG. 7), a server device 10 (refer to FIG. 7), and a subject terminal 40 (refer to FIG. 7). The transmit unit 166 also provides control to transmit determination results obtained by the determination unit 164 to, for example, the administrator terminal 30, the server device 10 (refer to FIG. 7), and the subject terminal 40. The transmit unit 166 also has a function of providing control to transmit answer data to questionnaires about changes in physical condition of the subject transmitted from the administrator terminal 30 to the administrator terminal 30, the server device 10 (refer to FIG. 7), and the subject terminal 40. The transmit unit 166 transmits various kinds of data to different destinations depending on functions of the transmit unit 166 and the communication environment. Specifically, when the transmit unit 166 is configured to transmit data to the administrator terminal 30 or the server device 10, the transmit unit 166 may transmit data to the subject terminal 40, and the subject terminal 40 may then transmit the data to the administrator terminal 30 or the server device 10.

The transmit unit 166 has a function of transmitting detection data (measurement data) of the individual detection sensors of the temperature and humidity sensor 120, the acceleration sensor 130, the thermopile sensor 140, and the pulse wave sensor 150 to external devices at every given time. The given time may be determined by, for example, a fixed length of intervals (for example one-minute intervals) or any lengths of intervals. The transmit unit 166 may change the given time depending on the remaining battery charge. For example, while the remaining battery charge exceeds 50%, detection data may be transmitted every one minute; while the remaining battery charge is 50% or less, detection data may be transmitted every ten minutes. The level of remaining battery charge and the transmission time point can be set at any level and any time point. The transmit unit 166 may accumulate a plurality of sets of detection data until data is transmitted and transmit the sets of detection data accumulated together at every given time. This configuration can reduce battery consumption. The detection data transmitted by the transmit unit 166 may be (1) actual measurement data measured by the individual detection sensors, (2) processed data generated by computationally processing the actual measurement data, or (3) both the actual measurement data and the processed data. Which kind of data is transmitted as detection data of the sensors may be changed depending on the function of the determination unit 164, the remaining battery charge, and other factors.

As described above, based on detection data of the individual detection sensors of the temperature and humidity sensor 120, the acceleration sensor 130, the thermopile sensor 140, and the pulse wave sensor 150, the physical condition change detection device 100 can determine whether a change in physical condition of the subject wearing the physical condition change detection device 100 has occurred (a risk that a change in physical condition can occur). The physical condition change detection device 100 can also exchange data relating to questionnaires about changes in physical condition of the subject wearing the physical condition change detection device 100 with the administrator terminal 30 (refer to FIG. 7) and the server device 10. Details of determination of whether a change in physical condition of the subject has occurred and management of changes in physical condition with the use of the physical condition change detection device 100 of the present embodiment will be described later.

How to Use Sensors of Physical Condition Change Detection Device 100

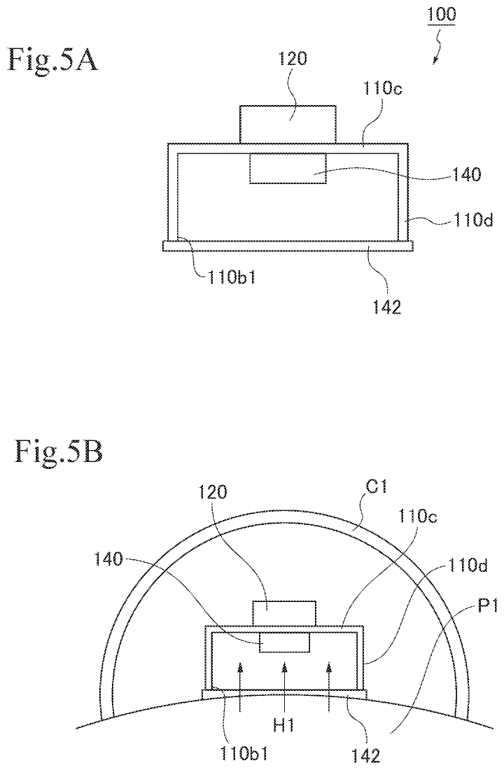

The following describes how to use sensors of the physical condition change detection device 100. FIG. 5(A) illustrates how to use sensors of the physical condition change detection device 100. FIG. 5(B) illustrates an operation of the physical condition change detection device 100. To clarify the position of the temperature and humidity sensor 120 and the position of the thermopile sensor 140 with respect to the substrate 110c and the position of the thermally conductive sheet 142 fixed to the cavity 110b1 on the back surface 110b side of the casing 110, FIGS. 5(A) and 5(B) provide simplified sectional views including only these elements.

In the physical condition change detection device 100, as illustrated in FIG. 5(A), the temperature and humidity sensor 120 is provided on the outer surface of the substrate 110c, and the thermopile sensor 140 is provided on the inner surface of the substrate 110c that is to face the body surface. To detect thermal variation in an outside area separated from the body surface of the subject, the temperature and humidity sensor 120 is provided on the outer surface of the substrate 110c. To detect radiant heat from the body surface of the subject, the thermopile sensor 140 is provided on the inner surface of the substrate 110c apart from the body surface and is to face the body surface.

The thermally conductive sheet 142 is provided at the cavity 110b1 on the lower end side of a side wall 110d of the substrate 110c. The thermally conductive sheet 142 is formed by fixing a black-body tape on a surface of a highly conductive sheet made of, for example, Cu or Al. Because the thermally conductive sheet 142 is provided at the cavity 110b1, it is possible to avoid malfunctions due to entry of sweat or liquid to the thermopile sensor 140. Additionally, this structure facilitates transfer of radiant heat to the thermopile sensor 140. Although the thermally conductive sheet 142 is provided at the cavity 110b1 in the present embodiment, the thermally conductive sheet 142 is not necessarily provided.

When the physical condition change detection device 100 formed in this manner is attached to, for example, an upper arm of the subject P1, as illustrated in FIG. 5(B), radiant heat H1 from the body surface of the subject is transferred through the thermally conductive sheet 142 to the thermopile sensor 140. The thermopile sensor 140 can detect variations in the radiant heat H1 radiated from the body surface of the subject. The temperature and humidity sensor 120 provided on the outer surface of the substrate 110c of the physical condition change detection device 100 can detect variations in the temperature and humidity of the air inside a garment C1 worn by the subject, as illustrated in FIG. 5(B). The garment C1 is not necessarily a piece of clothing of the subject and may be a holder made of cloth for holding the physical condition change detection device 100 on the body.

As described above, the thermopile sensor 140 detects variations in radiant heat from the body surface of the subject; by contrast, the temperature and humidity sensor 120 detects variations in the temperature and humidity in the nearest outside environment (the environment inside clothes) on the opposite side with respect to the thermopile sensor 140 across the substrate 110c. As a result, the physical condition change detection device 100 can accurately detect a risk that a change in physical condition such as heatstroke can occur based on thermal variation in the internal and external environments of the body of individual target people.

Arrangement of Sensors of Physical Condition Change Detection Device 100

The physical condition change detection device 100 is configured such that the thermopile sensor 140 serving as the “body-surface-side biological information detection unit” and the temperature and humidity sensor 120 serving as the “outside thermal variation detection unit” are provided individually on the back surface side and front surface side of the substrate 110c. However, the physical condition change detection device 100 may be configured in other manner. For example, the temperature and humidity sensor 120 illustrated in FIG. 5(A) and the thermopile sensor 140 may be disposed side by side.

For example, a substrate 210c2 having a temperature and humidity sensor 220 and a substrate 210c1 having a thermopile sensor 240 are not necessarily the same substrate and may be separate substrates as illustrated in FIG. 6(A). These substrates 210c1 and 210c2, which are separate substrates, are positioned with a difference in level. This structure reduces the thickness of a physical condition change detection device 200. In this manner, the thermopile sensor 240 is spaced apart from the body surface of the subject to face the body surface, and the temperature and humidity sensor 220 is provided on the surface opposite to the thermopile sensor 240 so that the temperature and humidity sensor 220 can detect thermal variation in an outside area separated from the body surface of the subject. This structure can achieve the same effect as the physical condition change detection device 100 according to an embodiment.

In the physical condition change detection device 200, lower end portions of side walls 210d1 and 210d2 of the substrate 210c1 having the thermopile sensor 240 form a cavity 210b1. A thermally conductive sheet 242 is fixed to the cavity 210b1. As described above, the substrate 210c1 having the thermopile sensor 240 and the substrate 210c2 having the temperature and humidity sensor 220 are arranged with a difference in level on the side. This structure reduces the thickness of the physical condition change detection device 200.

Alternatively, as illustrated in FIG. 6(B), the detection sensors provided on the outer and inner sides of the substrate 310c may be implemented as the thermopile sensors 340a and 340b. When the thermopile sensors 340a and 340b are provided on the front and back surface sides of the substrate 310c, it is necessary to fix the thermally conductive sheets 342a and 342b respectively to a cavity 310b1 provided at one end of a side wall 310d1 and a cavity 310b2 provided at one end of a side wall 310d2. Because the thermally conductive sheets 342a and 342b are respectively provided at the cavities 310b1 and 310b2, it is possible to block entry of sweat or liquid from surfaces on the sides for detection and increase conductivity of heat to the thermopile sensors 340a and 340b. A physical condition change detection device 300 is formed in this manner, and thus, based on the radiant heat from the body surface of the subject and the radiant heat in an outside area with respect to the physical condition change detection device 300, it is possible to detect changes in physical condition of the subject such as heatstroke in a reduced time.

Alternatively, as illustrated in FIG. 6(C), the detection sensors provided on the outer and inner sides of a substrate 410c may be implemented as temperature and humidity sensors 420a and 420b. When the temperature and humidity sensors 420a and 420b are provided on the front and back surface sides of the substrate 410c, to measure temperature and humidity, the temperature and humidity sensors 420a and 420b need to be positioned in direct contact with detection targets. As a result, it is unnecessary to position the temperature and humidity sensors 420a and 420b apart from the surfaces toward detection areas. This structure increases time to be taken to detect thermal variation but reduces the thickness of a physical condition change detection device 400.

Basic Configuration of Physical Condition Change Management System 1

The following describes a basic structure of a physical condition change management system according to an embodiment of the present invention with reference to the drawings. FIG. 7 is a block diagram illustrating a basic configuration of the physical condition change management system according to the present embodiment.

In a physical condition change management system 1 of the present embodiment, workers who work outside, workers who work inside, and other people who stay inside or outside to do some activities or live their life and need detection of changes in physical condition are all regarded as target people in need of detection of changes in physical condition such as heatstroke. The physical condition change management system 1 is used to determine, for example, whether a change in physical condition of the individual subject has occurred or is likely to occur by using the physical condition change detection device 100 and manage the physical condition of the individual subject.

The physical condition change management system 1 of the present embodiment includes, as illustrated in FIG. 7, the server device 10, a data storage unit 20, the administrator terminal 30, the subject terminal 40, and the physical condition change detection devices 100, which are connected to each other through the network 2, for example the Internet. The data storage unit 20 is an “external storage device” of the server device 10 and configured to be accessible by only the server device 10. The physical condition change detection device 100 is configured to be connectable to at least any of the server device 10, the subject terminal 40, and the administrator terminal 30. The administrator terminal 30 and the subject terminal 40 are “terminal devices” that can communicate with external devices through the network 2. In the present embodiment, the network 2 may be a wired or wireless network such as a local area network (LAN) or wide area network (WAN) or may be a dedicated line instead of a public network such as the Internet.

The server device 10 has a function of managing, for example, biological information including body temperature and heart rate received from a plurality of target people P1, P2, and P3, detection data of the physical condition change detection devices 100, and answer data to questionnaires about changes in physical condition of the target people P1, P2, and P3 in a computational manner and manage the physical condition of the individual target people P1, P2, and P3, including whether a change in physical condition of the individual target people P1, P2, and P3 has occurred or is likely to occur. This means that the server device 10 is a computer device configured to function as a “physical condition change management server”.

The server device 10 is managed by being used by an administrator who manages physical condition of target people in need of detection of changes in physical condition. The target people targeted for management of changes in physical condition with the physical condition change management system 1 may be, for example, workers who work outside, workers who work inside, and other people who stay inside or outside to do some activities. In the present embodiment, various computer devices such as a supercomputer, general-purpose computer, office computer, control computer, and personal computer may be used as the server device 10. Details of the server device 10 of the present embodiment will be described later.

The data storage unit 20 functions as a database for storing “objective data” and “subjective data” of the individual target people P1, P2, and P3. The “objective data” includes measurement data obtained by the detection sensors of the physical condition change detection device 100 used by the individual target people P1, P2, and P3 and processed data generated by computationally processing the measurement data. The “subjective data” includes answer data to a questionnaire answered by the target people P1, P2, and P3 before activity (before start of detection of changes in physical condition), answer data to a questionnaire answered by the target people P1, P2, and P3 during activity (during detection of changes in physical condition), and answer data to a questionnaire answered by the target people P1, P2, and P3 after activity (after detection of changes in physical condition). The “subjective data” also includes body information of the target people P1, P2, and P3, such as sex, age, and BMI, personally identifiable information, and other information. As such, in the present embodiment, the data storage unit 20 stores for the individual target people P1, P2, and P3 the objective data and subjective data that are used as the basis for determination of whether a change in physical condition has occurred or determination of a risk that a change in physical condition can occur.

The “objective data” of the individual target people P1, P2, and P3 stored in the data storage unit 20 include “labeled objective data” generated by attaching to the objective data a label determined depending on differences in heat that the target people feel (subjective data). Different people feel different heat depending on various factors such as the person's body size, physical constitution, sex, and age. Hence, when such a personal factor is added, the obtained determination results are more suitable to individual target people than when determination of changes in physical condition is provided based on only the “objective data (actual measurement data)” measured by the sensors of the physical condition change detection device 100. For this purpose, the “objective data” includes “labeled objective data”. In the present embodiment, a control unit 14 (a determination unit 14b) described later attaches a label (annotation) for differentiating changes in physical condition to the objective data (actual measurement data) based on the subjective data (questionnaire answer data), so that the labeled objective data is generated. This labeled objective data is usable as training data for machine learning. The labeled objective data is thus usable as data for determining by comparison whether the objective data measured by the sensors of the physical condition change detection device 100 for the individual target people indicates a risk of occurrence of a change in physical condition.

In the present embodiment, the data storage unit 20 is implemented by a cloud storage device configured to store data files in a disk space such as an external server through the network 2. The data storage unit 20 may be implemented by an external storage device directly connected to the server device 10 without using the network 2.

The administrator terminal 30 is a terminal device used by an administrator, who manages the target people P1, P2, and P3 targeted for detection of changes in physical condition, to manage physical condition when, for example, a change in physical condition has occurred in the target people P1, P2, and P3. The administrator terminal 30 is a computer device capable of performing various kinds of computational processing, for example a desktop or laptop personal computer, or a mobile information terminal such as a touch pad or smartphone. In the present embodiment, the administrator terminal 30 can access the server device 10, the subject terminal 40, the physical condition change detection device 100, and the data storage unit 20 through the network 2.

The subject terminal 40 is a terminal device used when the target people P1, P2, and P3 targeted for detection of changes in physical condition exchange data with the administrator terminal 30. The subject terminal 40 is a computer device capable of performing various kinds of computational processing, for example a desktop or laptop personal computer, or a mobile information terminal such as a touch pad or smartphone. In the present embodiment, the subject terminal 40 can communicate with the administrator terminal 30 through the network 2 to transmit the subjective data generated by collecting answers to questionnaires about physical condition of the individual target people P1, P2, and P3, personally identifiable information including sex, age, and BMI, and other information and to enable the administrator terminal 30 to provide a suggestion that the subject should take a rest due to a change in physical condition such as heatstroke.

Specific Configuration of Physical Condition Change Management System 1

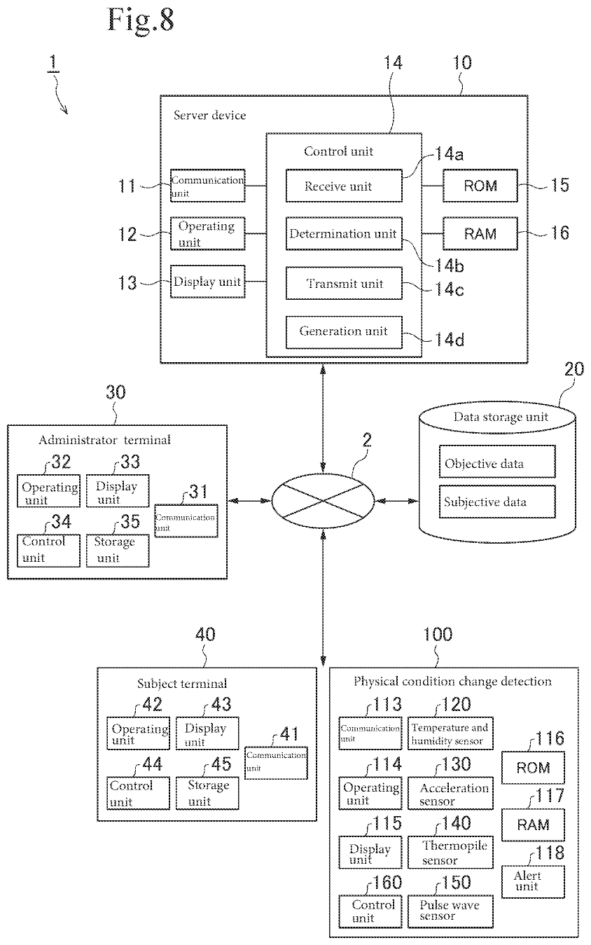

The following describes specifics of main parts of the physical condition change management system 1. FIG. 8 is a block diagram illustrating a specific configuration of main parts of the physical condition change management system 1. Referring to FIG. 8, of the physical condition change management system 1, only the server device 10, the data storage unit 20, the administrator terminal 30, and the subject terminal 40 and the physical condition change detection device 100 that are worn or used by the subject P1 will be described, and functions of individual elements will be described in detail.

In the physical condition change management system 1, the server device 10 is connected to the data storage unit 20, the administrator terminal 30, the subject terminal 40, and the physical condition change detection device 100 through the network 2. With this configuration, the server device 10 creates a database based on the objective data and subjective data received from the subject P1 and manages the database with the data storage unit 20, while the administrator using the administrator terminal 30 can monitor the physical condition of each subject.

Specific Configuration of Server Device 10

The server device 10 includes, as illustrated in FIG. 8, a communication unit 11, an operating unit 12, a display unit 13, the control unit 14, a ROM 15, and a RAM 16. The server device 10 implements the “physical condition change management server” having a plurality of functional units implemented by the control unit 14 running the “physical condition change management program”; the plurality of functional units perform information processing. The communication unit 11 functions as an interface when data is exchanged from the outside through the network 2. The operating unit 12 has a function as an input device for inputting data, such as a keyboard and a mouse, or a touch panel; the function is used to operate the server device 10 by inputting predetermined instructions to the control unit 14.

The display unit 13 has a function of outputting, for example, results of operations performed by the control unit 14 and information in the data storage unit 20, which is a database, by displaying. The display unit 13 may be implemented by, for example, a liquid crystal screen. The display unit 13 is configured to display a transition graph of objective data including the heart rate, momentum, and radiant heat of the subject wearing the physical condition change detection device 100.

The control unit 14 has a function of controlling operations of the elements included in the server device 10 by running various programs stored in the ROM 15 with one or a plurality of processors. The control unit 14 also has a function of, when providing control to execute these various operations, causing the RAM 16, which temporarily stores data, to store required data and other data as appropriate. As a result, control by the control unit 14 enables operations including an operation of accessing the ROM 15, the RAM 16, and the data storage unit 20, an operation of displaying data with the display unit 13, an operation on the operating unit 12, and an operation of exchanging various kinds of information using the communication unit 11 as an interface through the network 2 when communicating with the outside.

The control unit 14 includes, as illustrated in FIG. 8, a receive unit 14a, the determination unit 14b, a transmit unit 14c, and a generation unit 14d. The receive unit 14a has a function of providing control to receive various kinds of data from the data storage unit 20, the administrator terminal 30, the subject terminal 40, and the physical condition change detection device 100 via the communication unit 11. In the present embodiment, the receive unit 14a is controlled to receive objective data (actual measurement data and/or processed data) including the measured heart rate and body temperature of the subject P1 transmitted from the physical condition change detection device 100 to the server device 10 at every given time.

The determination unit 14b has a function of providing determination required when various operations of the server device 10 are performed. For example, the determination unit 14b determines whether various kinds of data are exchanged with the administrator terminal 30, the subject terminal 40, and the physical condition change detection device 100 via the communication unit 11 of the server device 10.

The determination unit 14b has a function of determining whether a change in physical condition has occurred in the subject P1 (whether there is a risk that a change in physical condition can occur), based on variations in biological information including at least the radiant heat from the body surface of the subject P1 detected by the physical condition change detection device 100 and thermal variations in an outside area with respect to the substrate 110c provided in the casing 110 of the physical condition change detection device 100. The determination unit 14b also has a function of determining a preliminary change in physical condition that is highly likely to proceed to heatstroke, with additional reference to answers to questionnaires about physical condition of the subject P1 transmitted from the administrator terminal 30 to the subject terminal 40 and the physical condition change detection device 100.

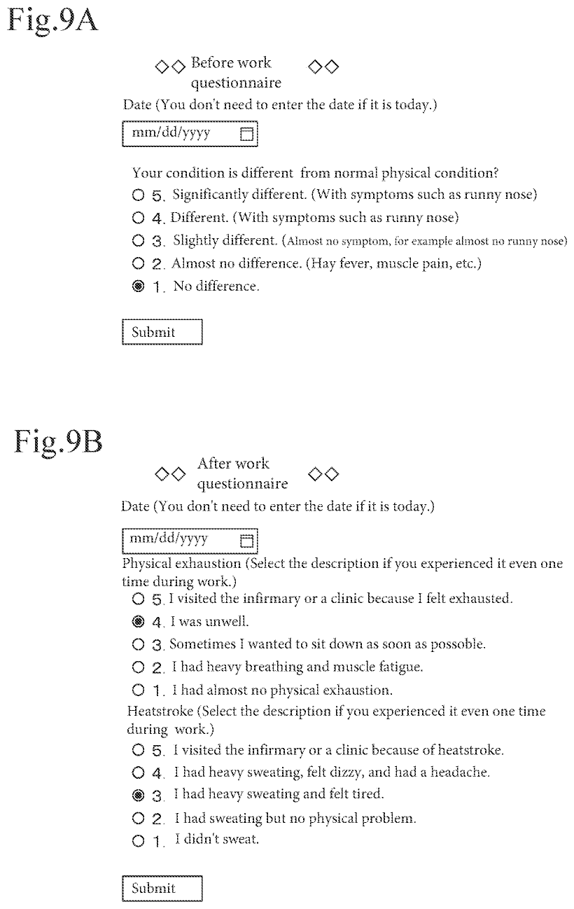

In the present embodiment, the determination unit 14b also has a function of generating labeled objective data (training data) by attaching a label (annotation) for differentiating changes in physical condition to objective data, based on questionnaire answer data from the subject P1. In an example, when the answer from the subject P1 indicates “1” or “2” in a before work questionnaire (FIG. 9(A)) and in an after work questionnaire (FIG. 9(B)) about “heatstroke”, the risk of developing heatstroke is determined to be low. The determination unit 14b accordingly attaches a label “not potential heatstroke group” to objective data of an activity date answered in the questionnaires. By contrast, when the answer from the subject P1 indicates “3”, “4”, or “5” in a before work questionnaire (FIG. 9(A)) and in an after work questionnaire (FIG. 9(B)) about “heatstroke”, it is determined that there is a risk of developing heatstroke. The determination unit 14b accordingly attaches a label “potential heatstroke group” to objective data of an activity date answered in the questionnaires. As such, the determination unit 14b functions as a classifier. By collecting the labeled objective data as “training data” and repeatedly performing machine learning with the labeled objective data, it is possible to improve the accuracy of determination of a risk that a physical condition problem can occur (a risk of developing heatstroke depending on different levels of heat that individual target people feel) for individual target people. The determination unit 14b compares the received objective data to the labeled objective data used in machine learning and determines whether a change in physical condition has occurred in the subject (a risk of developing heatstroke). The determination result obtained for the individual target people P1 with this configuration is more suitable than when determination of whether there is a risk of occurrence of a change in physical condition is based on only the “objective data” measured by the individual detection sensors of the physical condition change detection device 100.

The determination of a risk of occurrence of a change in physical condition by the determination unit 14b is not necessarily based on the labeled objective data generated by supervised machine learning. This means that, to determine whether biological information (vital signs) measured as objective data is data indicating a risk of occurrence of a change in physical condition in the subject, it is sufficient to use criteria (feature and its threshold) that are set especially for the subject in accordance with their physical characteristics. Thus, data of a particular kind of biological information (and a corresponding predetermined value) obtained as a feature by unsupervised machine learning may be used as the criteria. The criteria may be data generated by subjecting biological information to deep learning. Additionally, machine learning is not necessarily used when the determination unit 14b determines a change in physical condition of the subject P1. The determination unit 14b may determine whether a change in physical condition has occurred, by setting, for the objective data (measurement data) measured by the physical condition change detection device 100, a threshold at which a risk of occurrence of the change in physical condition is expected based on personal views or subjective data (questionnaire answer data) of the subject and using the threshold as comparison objective data for determining whether there is a risk.

The questionnaire answer data to the after work questionnaire (FIG. 9(B)) about “physical exhaustion” may be used, for example, as follows. The more easily a person feels exhausted due to physical exhaustion, the weaker their physical strength is. In this case, it is necessary to pay attention to a risk of occurrence of changes in physical condition. Thus, in the case in which a person is determined to have fatigue to be cared, for example when the answer data indicates “3”, “4”, or “5” in questions of the questionnaires, it is possible to send an email or notification for raising attention to the occurrence of a change in physical condition to the physical condition change detection device 100 and the subject terminal 40 on the next day. In the case in which the level of fatigue is determined to be relatively high, for example when the answer data indicates “3”, “4”, or “5”, a higher risk of occurrence of a change in physical condition is expected; based on this expectation, the labeled objective data is adjusted to set more specific criteria for determining a risk of occurrence of a change in physical condition on objective data.

The transmit unit 14c has a function of providing control to transmit various kinds of data to the data storage unit 20, the administrator terminal 30, the subject terminal 40, and the physical condition change detection device 100 via the communication unit 11. The transmit unit 14c has a function of transmitting determination results obtained by the determination unit 14b to at least one of the subject terminal 40 and the administrator terminal 30.

The transmit unit 14c also has a function of outputting an alert as a warning to an external device when the determination unit 14b determines that there is a risk of occurrence of a change in physical condition (a risk of occurrence of heatstroke). In this case, the transmit unit 14c can output an alert to at least any of the subject terminal 40, the administrator terminal 30, and the physical condition change detection device 100.

When the transmit unit 14c outputs an alert to the physical condition change detection device 100, the alert unit 118 of the physical condition change detection device 100 starts operation. When the alert unit 118 starts operation, the alert unit 118 outputs an alert indicating that the risk of developing heatstroke is relatively high by, for example, displaying a representation of the alert indicating the risk of occurrence of heatstroke with the display unit 115, providing a voice announcement about the alert, sounding an alarm, or producing vibrations. The transmit unit 14c also has a function of, when answer data to a sent questionnaire has not been sent to the physical condition change detection device 100, outputting an alert for requesting the subject P1 to answer the questionnaire. The transmit unit 14c may transmit changes in environment information or other information, which can cause, for example, changes in physical condition, as one kind of alert. For example, the transmit unit 14c may transmit as an alert a change in environment information that is likely to cause a physical condition change or problem, such as a photochemical smog warning or pollen or PM2.5 count information, so as to raise attention from the subject to a risk of a physical condition change or problem.

In the present embodiment, the transmit unit 14c also has a function of, when the determination unit 14b determines that a change in physical condition can occur in the subject P1, sending to the administrator terminal 30 an alert mail for providing a warning of a risk of occurrence of a change in physical condition such as heatstroke in the subject P1. Specifically, when the determination unit 14b determines, as a change in physical condition of the subject P1, that the subject P1 is at a risk of developing heatstroke, the transmit unit 14c sends an alert mail for providing a report about the risk from the subject P1 to the administrator terminal 30.

The generation unit 14d has a function of computationally processing various kinds of data and generating data. The generation unit 14d generates, for example, display data. Examples of the display data include objective data (actual measurement data) including the heart rate, momentum, and radiant heat of the subject measured by the physical condition change detection device 100, a transition graph of the objective data, and a table containing the objective data. The display data generated by the generation unit 14d can be transmitted to external devices (for example, the administrator terminal 30 and the subject terminal 40) via the communication unit 11. The external devices can display the display data as a display screen with a display unit such as a display.

The data storage unit 20 is an external storage device capable of storing various kinds of data. In the present embodiment, the data storage unit 20 functions as a database for storing, for each subject, the objective data obtained by sensing with the detection sensors of the physical condition change detection device 100 and the subjective data generated by previously collecting answer data to questionnaires about physical condition and body information and personally identifiable information including sex, age, and BMI. The data storage unit 20 is updated every time the objective data and the subjective data are updated.

Specific Configuration of Administrator Terminal 30

The administrator terminal 30 is a terminal device used by an administrator. To perform required operations such as operations of transmitting and receiving various kinds of information and computational processing, the administrator terminal 30 includes, as illustrated in FIG. 8, a communication unit 31, an operating unit 32, a display unit 33, a control unit 34, and a storage unit 35. By accessing the server device 10, the administrator terminal 30 can display with the display unit 33 the display data relating to objective data, including the objective data of the subject wearing the physical condition change detection device 100 containing heart rate, momentum, radiant heat, and other information, a transition graph of the objective data, and a table of the objective data.

For the subject P1, the administrator terminal 30 transmits the before work questionnaire (questionnaire data) about changes in physical condition of the subject P1 before work to both the subject terminal 40 and the physical condition change detection device 100 of the subject P1 via the communication unit 31. As indicated in FIG. 9(A), for example, as a questionnaire about changes in physical condition of the subject P1, the before work questionnaire is created such that, as to changes from the normal physical condition, in other words, differences from the normal time, an answer can be simply selected from the following five levels: “5. Significantly different.”, “4. Different.”, “3. Slightly different.”, “2. Almost no difference.”, and “1. No difference.” The answer data to this questionnaire is transmitted to the server device 10 and then stored in the data storage unit 20. The determination unit 14b of the server device 10 uses the answer data as subjective data to generate labeled objective data as described above.

For the subject P1, the administrator terminal 30 transmits the after work questionnaire (questionnaire data) about changes in physical condition of the subject P1 after work to both the subject terminal 40 and the physical condition change detection device 100 of the subject P1 via the communication unit 31. As illustrated in FIG. 9(B), for example, as a questionnaire about changes in physical condition of the subject P1, the after work questionnaire is created such that, as to changes in physical condition relating to physical exhaustion and changes in physical condition relating to heatstroke, an answer to each question can be simply selected from corresponding five levels. To select an answer to each question about changes in physical condition relating to physical exhaustion or heatstroke, an instruction suggests selecting a description if the subject has experienced what the description explains even one time during work. The answer data to this questionnaire is transmitted to the server device 10 and then stored in the data storage unit 20. The determination unit 14b of the server device 10 uses the answer data as subjective data to generate labeled objective data as described above.

When the administrator terminal 30 receives an alert mail for providing a warning of occurrence of a change in physical condition of the subject P1 such as heatstroke from the server device 10, the administrator terminal 30 enables checking of the transition of objective data of the subject P1 on the WEB. As the result of monitoring the transition of objective data of the subject P1, when it is determined that a change in physical condition of the subject P1 such as heatstroke is highly likely to occur, the administrator terminal 30 transmits an alert signal to the physical condition change detection device 100 of the subject P1 and transmits an alert mail to the subject terminal 40 via the communication unit 31. As described above, in the present embodiment, the likelihood of developing heatstroke is reduced by providing an alert for the subject P1 to suggest that the subject P1 should take a break.

Specific Configuration of Subject Terminal 40

The subject terminal 40 is a terminal device used by the subject P1. To perform required operations such as operations of transmitting and receiving various kinds of information and computational processing, the subject terminal 40 includes, as illustrated in FIG. 8, a communication unit 41, an operating unit 42, a display unit 43, a control unit 44, and a storage unit 45. In the present embodiment, for example, by accessing a dedicated website provided by the server device 10, the subject terminal 40 can display with the display unit 43 the display data relating to objective data, including the objective data of the subject wearing the physical condition change detection device 100 containing heart rate, momentum, radiant heat, and other information, a transition graph of the objective data, and a table of the objective data.

As described above, in the physical condition change management system 1, the server device 10 is connected to the data storage unit 20, the administrator terminal 30, the subject terminal 40, and the physical condition change detection device 100 through the network 2. The server device 10 controls operation of a physical condition management application service in which, based on the objective data and subjective data received from a plurality of target people, it is determined whether a change in physical condition has occurred in each subject, and, for example, a notification of a warning indicating that there is a risk of occurrence of a change in physical condition for each subject is managed in a computational manner. As a result, the server device 10 can manage in a collective manner the physical condition of all the target people P1, P2, and P3 targeted for physical condition management.

In particular, in the present embodiment, as described above, the physical condition change detection device 100 also includes the operating unit 114 implemented by, for example, the input buttons 114a or the touch panel 114b, configured to receive inputs of answer data to the questionnaires about changes in physical condition transmitted from the administrator terminal 30. As a result, while traveling to a work site, the subject P1 can easily input answer data to the questionnaire about changes in physical condition also with the physical condition change detection device 100 that the subject P1 wears and transmit the answer data to the administrator terminal 30 via the server device 10. As a result, with the physical condition change management system 1 of the present embodiment, the administrator who manages physical condition of the subject P1 discovers a risk of developing heatstroke before the subject P1 develops heatstroke, based on the subjective data and the objective data with additional reference to questionnaire answer data from the subject P1. Furthermore, with the physical condition change management system 1, the administrator outputs an alert to the subject P1, so that it is possible to previously reduce the risk of developing heatstroke.

The server device 10 of the physical condition change management system 1 of the present embodiment may be implemented by either software or hardware. When the server device 10 is implemented by software, the individual functions are implemented by the control unit 14, which is a central processing unit (CPU), running a program for operating the physical condition change management system 1. The program of the present embodiment may be stored in the ROM 15 included in the server device 10 or in a computer-readable non-transitory storage medium.

In the present embodiment, the program may be stored in an external storage device, read, and run by cloud computing. In this case, the objective data obtained by the detection sensors with the physical condition change detection device 100 and the subjective data obtained from the individual target people with the subject terminal 40 or the physical condition change detection device 100 before or after activity may be collected in the server device 10 in the cloud and subjected to data analysis by, for example, time-series analysis, cluster analysis, or artificial intelligence.

### Effects of Embodiment

The following describes effects of the physical condition change detection device 100 and the physical condition change management system 1 according to the present embodiment.

In the physical condition change detection device 100, the thermopile sensor 140 serving as the radiant heat detection unit for detecting variations in radiant heat from the body surface of the subject P1 is to face the body surface of the subject. The temperature and humidity sensor 120 serving as the outside thermal variation detection unit for detecting thermal variation in an outside area separated from the body surface of the subject P1 is provided on the opposite surface in the detection direction of the thermopile sensor 140. This means that in the physical condition change detection device 100 of the present embodiment, the thermopile sensor 140 for detecting radiant heat from the body surface of the subject P1 is provided on, of the substrate 110c disposed in the casing 110, a surface that is to face the body surface of the subject P1. In the physical condition change detection device 100, the opposite surface of the substrate 110c has the temperature and humidity sensor 120 for measuring the temperature and humidity in an outside area with respect to the substrate 110c to detect thermal variation in an outside area separated from the body surface of the subject P1. As a result, by analyzing the difference between the variation data about radiant heat from the body surface of the subject P1 and the variation data about the temperature and humidity of the air in an outside area with respect to the physical condition change detection device 100, it is possible to detect a change in physical condition of the subject P1 such as heatstroke.

For example, in the case in which the subject targeted for detection of changes in physical condition, who wears the physical condition change detection device 100, is a worker at a work site such as a construction site or building site, the thermopile sensor 140 senses variation data about radiant heat from the body surface of the subject P1. The temperature and humidity sensor 120 senses variation data about the temperature and humidity of the air in an environment outside the physical condition change detection device 100, more specifically, inside a work uniform that is the nearest outside environment to the body surface of the subject. As a result, by analyzing the difference between the variation data about radiant heat sensed by the thermopile sensor 140 and the variation data about the temperature and humidity in the nearest outside environment sensed by the temperature and humidity sensor 120, it is possible to detect a change in physical condition of the subject P1 such as heatstroke. In the case in which the subject targeted for detection of changes in physical condition is an elderly person who, for example, is bedridden in a hospital room or stays in a hospital room in need of nursing care, by analyzing the difference between the variation data about radiant heat sensed by the thermopile sensor 140 and the variation data about the temperature and humidity in the nearest outside environment sensed by the temperature and humidity sensor 120, it is possible to detect a change in physical condition of the subject P1 such as hypothermia.

In particular, the factors of changes in physical condition such as heatstroke, hypothermia, physical exhaustion, and overexertion can vary depending on the body size of the subject P1 based on BMI and other indicator, the physical constitution including easiness of sweating, the physical condition based on, for example, how much fatigue has accumulated, and the environment and condition in which the subject P1 is present, specified by, for example, the air temperature, humidity, and sunlight strength at the work site. The physical condition change detection device 100 of the present embodiment is worn by the subject P1 by attaching the casing 110 to, for example, an upper arm of the subject P1 using the belt 112. When the target people P1 wear the physical condition change detection device 100, the physical condition change detection device 100 can detect as detection data variations in the radiant heat from the body surface and variations in the temperature and humidity inside the garment C1 such as a work uniform, for the individual target people P1. By analyzing these kinds of detection data, it is possible to determine a risk of occurrence of a change in physical condition (a risk of occurrence of heatstroke) of the subject P1 based on the analysis data. This is a personal heat indicator measurable based on the radiant heat radiated from the body surface of the subject P1 and the temperature and humidity inside clothes, and thus, this personal heat indicator can be considered as the Wet Bulb Globe Temperature (WBGT) determined for the individual target people P1. The present inventors propose the concept of this novel personal heat indicator as the “personal Wet Bulb Globe Temperature (pWBGT)”. WBGT indicates a heat index determined for an environment such as an inside environment, whereas pWBGT indicates a heat index determined for an individual. This pWBGT can more accurately determine the risk of occurrence of a physical condition change or problem of the subject P1, and as a result, the administrator can properly manage the physical condition of the individual target people P1.

In the physical condition change detection device 100, as sensors for sensing biological information of the subject P1 and other information, the acceleration sensor 130 and the pulse wave sensor 150 are provided in addition to the temperature and humidity sensor 120 and the thermopile sensor 140. As a result, in addition to detection results of the temperature and humidity sensor 120 and the thermopile sensor 140, based on changes in the momentum of the subject sensed by the acceleration sensor 130 and changes in the heart rate sensed by the pulse wave sensor 150, it is possible to more accurately estimate the risk of occurrence of a change in physical condition of the subject P1 such as heatstroke.

Although the thermopile sensor 140 is separated from the body surface for the purpose of measuring radiant heat, it is necessary to avoid malfunctions caused by the contact of, for example, sweat or liquid with the thermopile sensor 140. For this reason, in the present embodiment, the thermally conductive sheet 142 is fixed to the cavity 110b1 at the back surface 110b of the casing 110. This structure prevents malfunctions caused by the contact of sweat or liquid such as water with the thermopile sensor 140. Additionally, while the thermally conductive sheet 142 formed by fixing a black-body tape facilitates absorption of radiant heat from the body surface, the radiant heat can be efficiently transferred to the thermopile sensor 140.

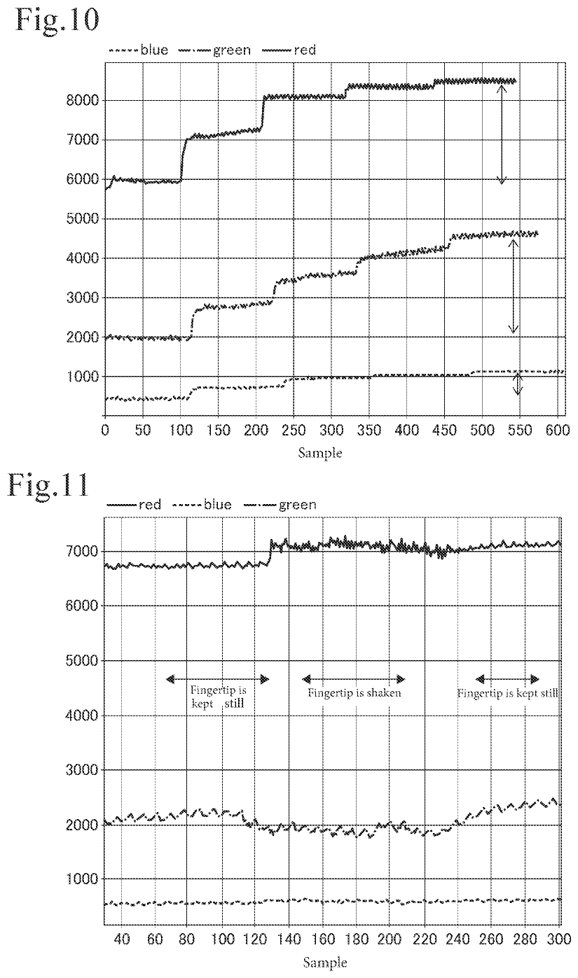

The pulse wave sensor 150, which measures the heart rate of the subject by emitting light with the color LED and receiving the light with the color sensor, is provided on the subject's body surface side of the casing 110. The behavior of the light emitted by the color LED changes when the wavelength of the light, that is, the color of the light changes. For example, as illustrated in FIG. 10, the waveform of a pulse wave of red measured with infrared light, indicates that while the light intensity increases by five levels, the fluctuation caused by pressure change, that is, the motion artifacts are relatively large. By contrast, the waveform of a pulse wave of blue measured with blue light, which has a relatively short wavelength, indicates that while the light intensity increases by five levels, the fluctuation caused by pressure change, that is, the motion artifacts are relatively small.

When the pulse wave sensor 150 implemented by a photoplethysmograph is fixed on the skin, because the distance between the skin and an artery varies depending on the fixing pressure, the attenuation of light seems different. The attenuation of light also seems different depending on differences among individual target people regarding, for example, body size or skin color. The distance between the pulse wave sensor 150 and an artery decreases when the pulse wave sensor 150 is tightly fixed. The distance increases when the pulse wave sensor 150 is loosely fixed. When the skin color is white, the attenuation of light decreases. When the skin color is brown, the attenuation of light increases.

In the state in which the photoplethysmograph as the pulse wave sensor 150 was fixed to a fingertip, as indicated in FIG. 11, when the fingertip was kept still, shaken, and then kept still, as indicated by the waveform of a pulse wave of red, only the waveform in the periods in which the fingertip was kept still is clearly illustrated; after the fingertip started shaking, motion artifacts of the same amplitude as the waveform of the pulse wave or larger were added to the waveform of the pulse wave. By contrast, the waveform of a pulse wave of blue indicates that the amplitude of the waveform of the pulse wave is smallest, but motion artifacts are relatively small.

In the present embodiment, when the pulse wave sensor 150 is fixed, the color LED changes the optimum wavelength (color) that most efficiently reaches an artery in the state in which the pulse wave sensor 150 is being fixed, and the wavelength is received by the color sensor. With this configuration, the pulse wave sensor 150 uses red or green while the subject is at rest; while the subject moves, the color is changed to, for example, blue, which has a relatively short wavelength, so that motion artifacts are canceled. As such, the pulse wave sensor 150 can be configured as a motion artifact resistant pulse wave sensor. In this case, it is preferable that while the subject moves, the color be changed in response to body motion by using, for example, the acceleration sensor 130.

With the physical condition change management system 1, the administrator can determine for each subject whether there is a risk of occurrence of a change in physical condition, based on the objective data detected by the physical condition change detection device 100, received from a plurality of target people, and the subjective data of each subject, obtained from the subject terminal 40 or the physical condition change detection device 100. The administrator can thus manage in a collective manner the physical condition of the individual target people P1, P2, and P3 managed by the administrator. The administrator previously provides a warning for the subject who is at a risk of occurrence of a change in physical condition, so that it is possible to reduce the likelihood of developing, for example, heatstroke, hypothermia, physical exhaustion, or overexertion. In particular, to the subject in the potential heatstroke group, the administrator sends an alert, for example, of a warning of heatstroke or for requesting an answer to a questionnaire, so that it is possible to reduce the likelihood of developing heatstroke.

The physical condition change detection device 100 includes the operating unit 114 for inputting answer data to the questionnaires about changes in physical condition transmitted from the administrator terminal 30. As a result, at any time before or after work, the target people P1, P2, and P3 can input answer data from the physical condition change detection device 100 and transmit the answer data to the administrator terminal 30.

In the present embodiment, in the physical condition change detection device 100, the transmit unit 166 is controlled to transmit at least one of the detection data obtained by the detection sensors of the physical condition change detection device 100 and the processed data generated by computationally processing the detection data to external devices such as the administrator terminal 30 and the server device 10 at every given time. As a result, the administrator can monitor changes in physical condition of the subject while checking the detection data and the processed data generated by computationally processing the detection data. It is thus possible to previously reduce the likelihood of occurrence of a physical condition problem such as heatstroke.

In the present embodiment, the physical condition change detection device 100 further includes the alert unit 118 for notifying the subject of information. As a result, the subject can receive as alerts information including changes in physical condition of the subject, predictions about changes in physical condition, and environment information about an outside environment in which the subject is present. As a result, the subject can understand the risk of occurrence of physical condition problems based on changes in physical condition of the subject or changes in environment information. The subject can prevent the occurrence of physical condition problems by, for example, not going outside or taking a rest with hydration.

The generation unit 14d of the server device 10 generates display data. Examples of the display data include objective data (actual measurement data), a transition graph of the objective data, and a table containing the objective data. As a result, the subject terminal 40 and the administrator terminal 30 can display the objective data in various forms with the display units 43 and 33.

The physical condition change management system 1 can previously notify both the administrator and the subject of the risk of occurrence of a physical condition problem such as heatstroke before a change in physical condition occurs in the subject wearing the physical condition change detection device 100, and thus, the occurrence of a physical condition problem can be prevented. By monitoring the objective data sensed by the detection sensors of the physical condition change detection device 100, including the heart rate, momentum, body surface temperature, and pWBGT of the subject, and performing personal data analysis by using an AI function, it is possible to predict physical condition problems of the subject. As a result, more accurate determination results about changes in physical condition can be obtained, and the administrator can more properly manage the physical condition of the subject.

Although the embodiments of the present invention have been described in detail above, it can be understood by those skilled in the art that many kinds of modifications may be made without substantially departing from the new matter and effects of the present invention. These modifications are thus all embraced in the scope of the present invention.

For example, any term cited with a different term having a broader meaning or the same meaning at least once in the specification and the drawings can be replaced by the different term in any place in the specification and the drawings. The configuration and operation of the physical condition change detection device and the physical condition change management system are not limited to the configuration and operation described in the embodiments of the present invention, and various embodiments can be implemented.

### REFERENCE SIGNS LIST

1 physical condition change management system
2 network
10 server device
11, 31, 41 communication unit
12, 32, 42 operating unit
13, 33, 43 display unit
14, 34, 44 control unit
14a receive unit
14b determination unit
14c transmit unit
14d generation unit
15 ROM
16 RAM
20 data storage unit
30 administrator terminal
35, 45 storage unit
40 subject terminal
100, 200, 300, 400 physical condition change detection device
110 casing
110a front surface
110b back surface
110b1, 210b1, 310b1, 310b2 cavity
110c, 210c1, 210c2, 310c, 410c substrate
110d, 210d1, 210d2, 210d3, 310d1, 310d2 side wall
111 slit
112 belt
113 communication unit
114 operating unit
114a operating button
114b touch panel
115 display unit
116 ROM
117 RAM
118 alert unit
120 temperature and humidity sensor (outside thermal variation detection unit)
130 acceleration sensor
140 thermopile sensor (body-surface-side biological information detection unit, radiant heat detection unit)
142 thermally conductive sheet
150 pulse wave sensor (body-surface-side biological information detection unit)
160 control unit
162 receive unit
164 determination unit
166 transmit unit

1. A physical condition change detection device for detecting a change in physical condition of a subject, comprising:
a body-surface-side biological information detection unit configured to detect a variation in biological information obtained from a body surface of the subject; and
an outside thermal variation detection unit configured to detect a thermal variation in an outside area separated from the body surface of the subject.

2. The physical condition change detection device according to claim 1, wherein the body-surface-side biological information detection unit is a radiant heat detection unit configured to detect a variation in radiant heat from the body surface of the subject.

3. The physical condition change detection device according to claim 2, wherein the radiant heat detection unit is a thermopile sensor provided apart from the body surface.

4. The physical condition change detection device according to claim 1, wherein
the outside thermal variation detection unit is a temperature and humidity sensor configured to detect at least a variation in temperature and humidity in an outside area with respect to the body surface of the subject.

5. The physical condition change detection device according to claim 4, wherein the temperature and humidity sensor is configured to detect at least a variation in temperature and humidity inside a garment worn by the subject.

6. The physical condition change detection device according to claim 1, further comprising:
a transmit unit configured to provide control to transmit at least one of detection data of the body-surface-side biological information detection unit and the outside thermal variation detection unit and processed data generated by computationally processing the detection data to an external device at every given time.

7. The physical condition change detection device according to claim 1, wherein the body-surface-side biological information detection unit includes a pulse wave sensor configured to measure a heart rate of the subject.

8. The physical condition change detection device according to claim 1, further comprising:
an operating unit configured to input at least subjective data of the subject.

9. The physical condition change detection device according to claim 1, further comprising:
an alert unit configured to provide an alert including the change in physical condition for the subject.

10. A physical condition change management program causing at least one processor to function as at least a receive unit and a determination unit, wherein
the receive unit is configured to obtain measurement data indicating a variation in biological information obtained from a body surface of a subject and a thermal variation in an outside area separated from the body surface, and
the determination unit is configured to determine whether there is a risk of occurrence of a change in physical condition of the subject based on at least the measurement data.

11. The physical condition change management program according to claim 10, further causing the processor to function as a transmit unit, wherein
the transmit unit is configured to transmit a determination result obtained by the determination unit to at least an external device.

12. The physical condition change management program according to claim 10, wherein
the receive unit is configured to obtain subjective data about a physical condition that the subject feels, and
the determination unit is configured to determine whether there is the risk of occurrence based on the measurement data and the subjective data.

13. The physical condition change management program according to claim 12, wherein the subjective data includes answer data to a questionnaire about changes in physical condition of the subject.

14. The physical condition change management program according to claim 13, wherein the determination unit is configured to execute annotation processing for differentiating changes in physical condition with respect to the objective data based on the response data for each subject based on the answer data, and to generate labeled objective data for determining by comparison in physical condition specific to each subject.

15. The physical condition change management program according to claim 10, further causing the processor to function as a generation unit, wherein the generation unit is configured to generate display data including the measurement data.

16. A non-transitory computer-readable storage medium storing the physical condition change management program according to claim 10.

17. A physical condition change management system including at least one processor and a physical condition change management program causing the processor to function as at least a receive unit and a determination unit, wherein
the receive unit is configured to obtain measurement data indicating a variation in biological information obtained from a body surface of a subject and a thermal variation in an outside area separated from the body surface, and
the determination unit is configured to determine whether there is a risk of occurrence of a change in physical condition of the subject based on at least the measurement data.
